(19) United States
(12) Patent Application Publication
(10) Pub. No.: US 2005/0228325 A1
(43) Pub. Date: Oct. 13, 2005

(54) Support frame for relieving the vertebral column

(76) Inventors: Claudia Zours, Bochum (DE); Gerd-Peter Koppetsch, Dusseldorf (DE); Robert Kramer, Dusseldorf (DE)

Correspondence Address: WILLIAM COLLARD;COLLARD & ROE, P.C., 1077 NORTHERN BOULEVARD, ROSLYN, NY, 11576, US

(21) Appl. No.: 10/507,261

(22) PCT Filed: Mar. 10, 2003
(86) PCT No.: PCT/EP03/02430

§ 371 Date: May 16, 2005

(30) Foreign Application Priority Data
Mar. 12, 2002 (DE) 102 10 775.0
Mar. 12, 2002 (DE) 202 04 747.4

(51) Int. Cl. A61F 5/00

(52) U.S. Cl. 602/19

(57) ABSTRACT

The invention relates to a support frame for relieving the vertebral column in the trunk area between the hips and the thoracic vertebra, comprising a hip clasp supported on the hips of the patient and a thoracic vertebra clasp supported on the area of the thoracic vertebra, which are connected by means of rods extending parallel to the vertebral column. According to the invention, the distance between the hip clasp and the thoracic vertebra clasp can be adjusted so as to better adapt said support frame to the individual size of patients. For said purpose, the length of the support rods can be changed, preferably due to the fact that they are subdivided, wherein the support sections overlap in the longitudinal direction of the vertebral column and can be adjustably fixed to one another in the area of overlapping.

The invention relates to a support frame for relieving the vertebral column in the trunk area between the hips and the thoracic vertebrae, comprising a hip clasp supported on the hips of the patient and a thoracic vertebrae clasp supported on the area of the thoracic vertebrae, which are connected with one another by means of support rods extending parallel to the vertebral column.

Such support frames are used, for example, in connection with vertebral column ortheses or so-called bridging corsets, for post-operative treatment of patients suffering from problems with the vertebral column.

In the case of the known support frames of the aforementioned type, the hip clasps, the thoracic vertebrae clasps, and the support rods are made of thermoplastic plastic or of aluminum, and are connected with one another in one piece.

A problem in the case of such support frames is the adaptation to the individual body dimensions of the patient. It is true that it is known and easily possible to adapt the hip clasp and the thoracic vertebrae clasp to the individual body shape of the patient by means of bending or after heating of the thermoplastic plastic. However, it is not possible to adapt it to different patient heights in this manner. It is therefore necessary to keep many back support frames having different dimensions on hand in order to properly care for the patients, or to make them to measure.

It is therefore the task of the invention to create a support frame of the type stated, which can be better adapted to the individual height of the patient.

The object of the invention is a support frame for relieving the vertebral column in the trunk area between the hips and the thoracic vertebrae, comprising a hip clasp supported on the hips of the patient and a thoracic vertebrae clasp supported on the area of the thoracic vertebrae, which are connected with one another by means of support rods extending parallel to the vertebral column, whereby this support frame is characterized in that the distance between the hip clasps and the thoracic vertebrae clasp is adjustable.

The invention proceeds from the recognition that it is sufficient to adjust the distance between the hip clasp and the thoracic vertebrae clasp, in order to adapt the support frame to the height of the patient. In comparison, the hip clasp and the thoracic vertebrae clasp themselves can easily be adapted to different body shapes, particularly to the body circumference of the patient, because of their deformability. Therefore the support frame according to the invention as a whole can easily be adapted to all the individual body dimensions and body shapes of the patient. It is therefore sufficient to keep only a few support frame sizes on hand in order to properly care for patients.

A first advantageous embodiment of the invention provides that the support rods that run parallel to the vertebral column are adjustable in length.

It is practical if the length-adjustable support rods of the support frame are configured to be subdivided, whereby the support rod sections overlap, seen in the longitudinal direction of the vertebral column, and can be adjustably fixed to one another in the area of overlapping. In this case, the support rod sections are rigidly connected with the hip clasp and the thoracic vertebrae clasp, respectively, possibly made in one piece with them, and provided with means of attachment in the area of overlapping, with which they can be fixed to one another.

Particular advantages result if the support rod sections can be fixed to one another by means of hook and loop closures, in the area of overlapping. Such hook and loop closures have the advantage that they can be handled in particularly simple manner, and can transfer sufficiently great forces in the longitudinal direction of the support rods, whereby the support rod sections can be adjusted relative to one another in almost infinite manner.

Instead of the hook and loop closures, snaps, adhesive strips, or hooks and eyes can also be used.

If necessary, the support rod sections can also be guided on one another in telescoping manner, and can be fixed to one another at different extension lengths. It is also practical if these telescoping support rod sections are rigidly connected or made in one piece with the hip clasp and the thoracic vertebrae clasp, respectively.

Another advantageous embodiment of the support frame according to the invention provides that the support rods that run parallel to the vertebral column can be releasably attached both to the hip clasp and to the thoracic vertebrae clasp, and can be replaced with support rods having a different length and/or stiffness. The interchangeable support rods can be fixed in place on the hip clasp, on the one hand, and on the thoracic vertebrae clasp, on the other hand, by means of hook and loop closures, snaps, adhesive connectors, hooks and eyes, or the like, for example.

Preferably, however, it is provided that accommodation pockets are provided on the hip clasp, on the one hand, and on the thoracic vertebrae clasp, on the other hand, into which support rods having different lengths can be inserted. In this case, of course, the support rods are not an integral part of the hip clasp and the thoracic vertebrae clasp, respectively, but rather separate parts, separated from them. In this case, support rods having different lengths must be kept on hand in order to adapt the support range of the support frame, and they must be selected in accordance with the body dimensions of the patient. Alternatively, the interchangeable support rods can also be configured in such a manner that they can be shortened to the correct length, in each instance, using simple aids, for example a suitable cutting tool.

Such support rods, which can be inserted into accommodation pockets, can also be used in addition to the length-adjustable support rods explained initially, which are rigidly connected with the hip clasp and the thoracic vertebrae clasp, respectively. By means of these additional support rods, inserted into accommodation pockets, the support effect of the support frame can be reinforced, if necessary. Furthermore, there is the possibility of configuring the support frame to be stiffer or more flexible, by means of interchangeable support rods having a different thickness. In this way, it is possible to wean the patient from the orthesis in steps, by first using more rigid support rods, then more flexible ones.

The support rods can consist of a suitable stiff plastic and/or of steel. Such support rods, reinforced with steel or made entirely of steel, have a particularly great stiffness at low weight and small external dimensions. The latter is particularly important so that the support frame, which might be worn under the clothing, does not take up too much space.

In the case that the support frame according to the invention is additionally supposed to be used to relieve the thoracic vertebral column, it is furthermore provided that the thoracic vertebrae clasp is provided with an extension that reaches all the way under the shoulder blades of the patient, which is also held by the support rods, which in this case are configured to be correspondingly longer. Using such an extension of the thoracic vertebrae clasp, it is possible to support the thoracic vertebrae region of the vertebral column, in addition to the lumbar region.

Exemplary embodiments of the invention will be explained in the following, using the drawings. These show:

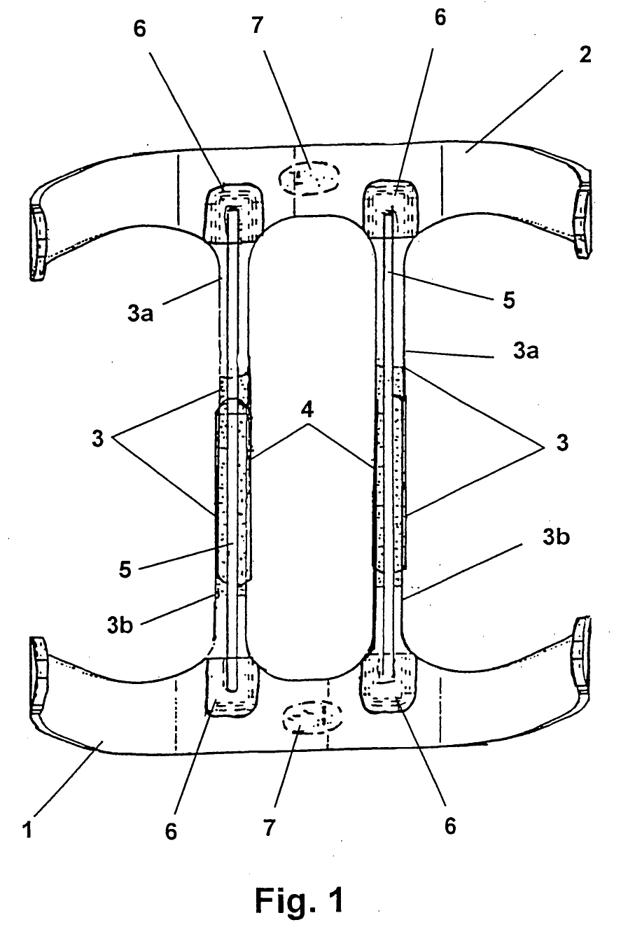

FIG. 1: A support frame according to the invention, in a first exemplary embodiment;

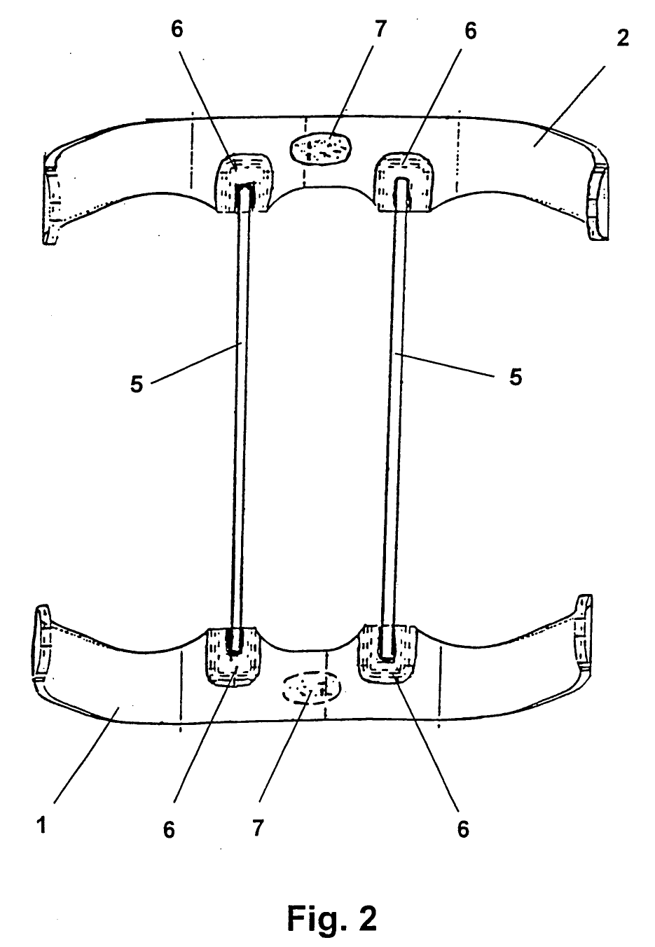

FIG. 2: A support frame according to the invention, in a second exemplary embodiment;

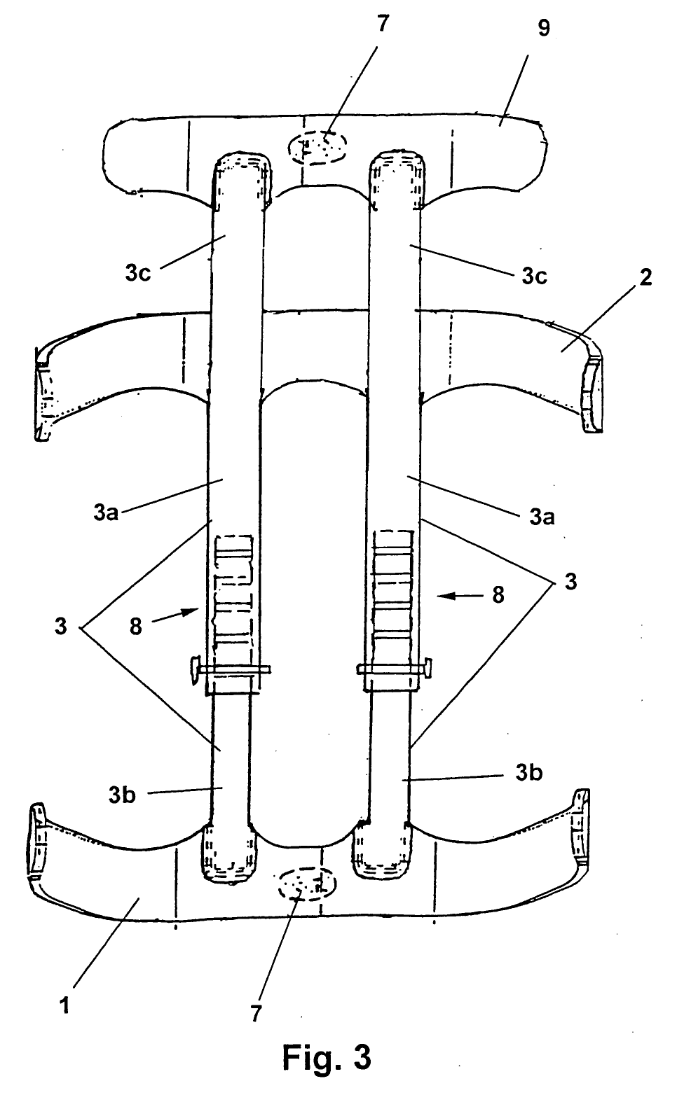

FIG. 3: A support frame according to the invention, in a third exemplary embodiment.

The support frame shown in FIG. 1 has a hip clasp 1 supported on the hips of the patient and a thoracic vertebrae clasp 2 that supports the back in the region of the thoracic vertebral column, which are connected with one another by means of support rods 3 that run parallel to the vertebral column. These support rods 3 of the support frame are subdivided, in each instance, whereby the support rod sections 3a and 3b overlap, seen in the longitudinal direction of the vertebral column, and can be fixed to one another in the area of overlapping by means of hook and loop closures 4. In this manner, the support rods 3 are adjustable in length and thereby the support range of the support frame can be adjusted.

For additional reinforcement or, if necessary, as a substitute for the aforementioned support rods 3, interchangeable support rods 5 are additionally provided, which can consist of steel, for example, and can be inserted into accommodation pockets 6 that are located on the hip clasp 1, on the one hand, and on the thoracic vertebrae clasp 2, on the other hand. The interchangeable support rods 5 can also be attached to the hip clasp 1, on the one hand, and to the thoracic vertebrae clasp 2, on the other hand, by means of other releasable means of attachment, if necessary.

The entire back support frame consists of thermoplastic plastic, so that it can be plastically deformed by means of heating it, in order to be able to adapt it to the anatomy of the patient. Alternatively, a different deformable material would also be possible.

In the case of the exemplary embodiment according to FIG. 2, the hip clasp 1 and the thoracic vertebrae clasp 2 are connected by means of interchangeable support rods 5 made of steel, which correspond to the interchangeable support rods 5 according to FIG. 1 and are inserted into accommodation pockets 6 that are located on the hip clasp 1, on the one hand, and on the thoracic vertebrae clasp 2, on the other hand. Here, the distance between the hip clasp 1 and the thoracic vertebrae clasp 2 can be changed in that support rods 5 having different lengths are inserted into the accommodation pockets 6. A similar procedure is also used in the case of the exemplary embodiment according to FIG. 1, if the interchangeable reinforcement rods 5 are supposed to be adapted to the length of the length-adjustable support rods 3 that has been set, in each instance.

In the case of the exemplary embodiment according to FIG. 3, the support rod sections 3a and 3b that are connected, in one piece, with the hip clasp 1 and the thoracic vertebrae clasp 2, respectively, are guided on one another in telescoping manner and can be fixed to one another at different extension lengths. The latter, in the case of this embodiment, takes place by means of the hole/plug connection indicated as 8.

In addition, in the case of this exemplary embodiment, the support rods 3 are extended beyond the thoracic vertebrae clasp 2 by means of support rod sections 3c that carry a support element 9 arranged below the shoulder blades of the patient. This support element 9 supports the vertebral column above the thoracic vertebrae clasp, in the region of the thoracic vertebral column. This support element 9 would also be possible in the case of the exemplary embodiments 1 and 2.

In the case of all three exemplary embodiments, the hip clasp 1 and/or the thoracic vertebrae clasp 2 are furthermore provided with hook and loop closures 7, with which the back support frame can be fixed to bandages or bridging corsets, which are not shown in the drawing.

1. Support frame for relieving the vertebral column in the trunk area between the hips and the thoracic vertebrae, comprising a hip clasp supported on the hips of the patient and a thoracic vertebrae clasp supported on the area of the thoracic vertebrae, which are connected with one another by means of support rods extending parallel to the vertebral column,
wherein
the distance between the hip clasp (1) and the thoracic vertebrae clasp (2) is adjustable.

2. Support frame as recited in claim 1, wherein the support rods (3) that run parallel to the vertebral column are adjustable in length.

3. Support frame as recited in claim 1, wherein the length-adjustable support rods (3) of the support frame are subdivided, in each instance, whereby the support rod sections (3a, 3b) overlap, seen in the longitudinal direction of the vertebral column, and can be adjustably fixed to one another in the area of overlapping.

4. Support frame as recited in claim 1, wherein the support rod sections (3a, 3b) are connected in one piece with the hip clasp (1) and the thoracic vertebrae clasp, (2) respectively, and provided with means of attachment in the area of overlapping, with which they can be fixed to one another.

5. The support rod sections (3a, 3b) can be fixed to one another by means of hook and loop closures (4), in the area of overlapping.

6. Support frame as recited in claim 1, wherein the support rod sections (3a, 3b) are guided on one another in telescoping manner, and can be fixed to one another at different extension lengths.

7. Support frame as recited in claim 1, wherein the support rods (5) that run parallel to the vertebral column can be releasably attached both to the hip clasp (1) and to the thoracic vertebrae clasp (2), and can be replaced with support rods (5) having a different length and/or stiffness.

8. Support frame as recited in claim 7, wherein accommodation pockets (6) are provided on the hip clasp (1), on the one hand, and on the thoracic vertebrae clasp (2), on the other hand, into which support rods (5) having different lengths can be inserted.

9. Support frame as recited in claim 1, wherein the length-adjustable support rods (3) and the interchangeable reinforcement rods (5) consist of plastic and/or of steel.

10. Support frame as recited in claim 1, wherein the thoracic vertebrae clasp (2) is additionally provided with an extension (3c, 9) that reaches all the way under the shoulder blades of the patient, which is also held by the support rods (3), which in this case are configured to be correspondingly longer.
